(19) United States
(12) Patent Application Publication
(10) Pub. No.: US 2014/0134684 A1
(43) Pub. Date: May 15, 2014

(54) Methods For Obtaining Oil From Maize Using Acid Protease and Cell-wall Polysaccharide-degrading Enzymes

(71) Applicant: The United States of America, as represented by the Secretary of Agriculture, (US)

(72) Inventors: David Johnston, Wyndmoor, PA (US); Kevin B. Hicks, Malvern, PA (US); Robert A. Moreau, Quakertown, PA (US); Jayarama K. Shetty, Pleasanton, CA (US)

(73) Assignee: The United States of America, as represented by the Secretary of Agriculture, Washington, DC (US)

(21) Appl. No.: 13/793,005

(22) Filed: Mar. 11, 2013

Related U.S. Application Data
(60) Provisional application No. 61/724,458, filed on Nov. 9, 2012.

(51) Int. Cl. C12P 7/64

(52) U.S. Cl. CPC C12P 7/64
USPC 435/134

(57) ABSTRACT

Disclosed are methods for obtaining oil from maize, involving grinding maize kernels to form flour, adding water to the flour to form a slurry, and incubating the slurry with α-amylase for about 10 minutes to about 180 minutes at a temperature of about 75° to about 120° C. and at a pH of about 3 to about 7 to form a mash, cooling the mash to about 15° C. to about 40° C. and adding a nitrogen source, glucoamylase, yeast, acid protease, and cell-wall polysaccharide-degrading enzymes to form a beer containing ethanol and oil, wherein the beer has a pH of about 3 to about 7, and recovering oil from the beer.

### REFERENCE TO RELATED APPLICATION

This application claims the benefit of U.S. Provisional Application No. 61/724,458, filed 9 Nov. 2012, which is incorporated herein by reference in its entirety.

### BACKGROUND OF THE INVENTION

Disclosed are methods for obtaining oil from maize, involving grinding maize kernels to form flour, adding water to the flour to form a slurry, and incubating the slurry with α-amylase for about 10 minutes to about 180 minutes at a temperature of about 75° to about 120° C. and at a pH of about 3 to about 7 to form a mash, cooling the mash to about 15° C. to about 40° C. and adding a nitrogen source, glucoamylase, yeast, acid protease, and cell-wall polysaccharide-degrading enzymes to form a beer containing ethanol and oil, wherein the beer has a pH of about 3 to about 7, and recovering oil from the beer.

There are two primary types of corn processing conducted presently: dry grind and wet milling processes. The wet milling processes are efficient in their use of corn since they produce numerous high value corn products, such as corn oil, starch, corn gluten meal, corn gluten feed, and corn steep liquor. However, the wet milling processes require very high capital investments in machinery. Dry grind ethanol processes are used to produce ethanol and animal feed. Animal feed is substantially less valuable than corn oil and zein, which are left in the animal feed produced by the dry grind process.

The recovery of post fermentation corn oil (often referred to as back-end oil recovery) by centrifugation of the concentrated thin stillage stream (syrup) has increased significantly in the last few years. The U.S. Environmental Protection Agency (EPA) has estimated that more than 60% of the dry grind ethanol producers will have adopted the technology by 2013 (EPA, 2011, Regulation of fuels and fuel additives: 2012 renewable fuel standards, Federal Register 76:38844-38890). This rapid implementation by the industry is due to the favorable economic payback as well as the minimal disruption to the existing ethanol process. The oil recovered using this technology is of relatively low quality due to the high free fatty acid levels which renders it undesirable for refining into food grade oil (Winkler-Moser, J. K., and L. Breyer, Industrial Crops and Products, 33: 572-578 (2011); Moreau, R. A., et al., Journal of the American Oil Chemists' Society, 87:895-902 (2010)). It is currently used as a feedstock for bio-diesel production and as an animal feed component.

While the technology has achieved rapid success in the industry, it also suffers from a number of significant technical issues. The primary issue is the low recovery yield. While some facilities report achieving better yields, most only recover about 0.25 kg per bushel (25.4 kg) of corn. This represents about 25% of the oil present in the incoming corn, which typically is about 4% oil on a dry weight basis (Johnston, D. B., et al., Journal of the American Oil Chemists' Society, 82:603-608 (2005); Moreau, R. A., et al., Journal of the American Oil Chemists' Society, 86:469-474 (2009)). The use of emulsion breakers as well as mechanical and thermal treatments has been found to be beneficial at improving yields in some facilities; however, recovery is still typically less than 30% and these additions increase the operational cost.

We have found that addition of acid protease and cell-wall polysaccharide-degrading enzymes (e.g., cellulases and hemicellulases) during fermentation increased post fermentation oil recovery, and that other processing factors (e.g., corn flour particle size) affected the oil recovery yields in a dry grind corn ethanol process.

### SUMMARY OF THE INVENTION

Disclosed are methods for obtaining oil from maize, involving grinding maize kernels to form flour, adding water to the flour to form a slurry, and incubating the slurry with α-amylase for about 10 minutes to about 180 minutes at a temperature of about 75° to about 120° C. and at a pH of about 3 to about 7 to form a mash, cooling the mash to about 15° C. to about 40° C. and adding a nitrogen source, glucoamylase, yeast, acid protease, and cell-wall polysaccharide-degrading enzymes to form a beer containing ethanol and oil, wherein the beer has a pH of about 3 to about 7, and recovering oil from the beer.

This summary is provided to introduce a selection of concepts in a simplified form that are further described below in the detailed description. This summary is not intended to identify key features or essential features of the claimed subject matter, nor is it intended as an aid in determining the scope of the claimed subject matter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows enzyme screening study results showing free oil recoveries for five enzyme preparations and the control (no enzyme addition) as described below. Enzymes were added at 10 kg enzyme/MT dry corn.

FIG. 2 shows free oil recovery using SPEZYME® CP as described below. Error bars represent ±one standard deviation of the duplicate average. Inset samples represent the actual amounts of free oil recovered from each 400 g mash using the dosage of SPEZYME® CP indicated.

FIG. 3 shows free oil recovery using GC 220 (square) and FERMGEN™ (circle). Error bars represent ±one standard deviation of the duplicate average as described below.

FIG. 4 shows particle size distribution of the corn flours used to study the effects on oil recovery as described below. Results shown are the averages of duplicate measurements.

FIG. 5 shows free oil recovery for corn flours prepared to study particle size effects on free oil recovery as described below. Degerminator ground (D), Coarse Ground (C), Medium Ground (M), Fine Ground (F), Polytron Ground (P) and GC 220 addition (+) at 10 kg enzyme/MT dry corn.

FIG. 6 shows free oil recovery for different ratios of GC 220 and FERMGEN™ at a fixed total enzyme level of 7 kg enzyme/MT dry corn as described below. Error bars represent ±one standard deviation of the duplicate average.

FIG. 7 shows free oil recovery for FERMGEN™ at 1.0 kg enzyme/MT dry corn and GC 220 at 2.5 kg enzyme/MT dry corn and the mixture of FERMGEN™ and GC 220 at the same levels as described below. Error bars represent ±one standard deviation of the triplicate average.

FIG. 8 shows the free oil recovery from the use of GC220 and FERMGEN™ individually and for the 2.5:1 mixture of GC220 and FERMGEN™ relative to the enzyme dose as described below.

FIG. 9 shows the free oil recovery using different ratios of GC220 to FERMGEN™ at equal enzyme doses of 2 kg/MT as described below.

FIG. 10 shows a general process model for the corn dry grind ethanol process as described below.

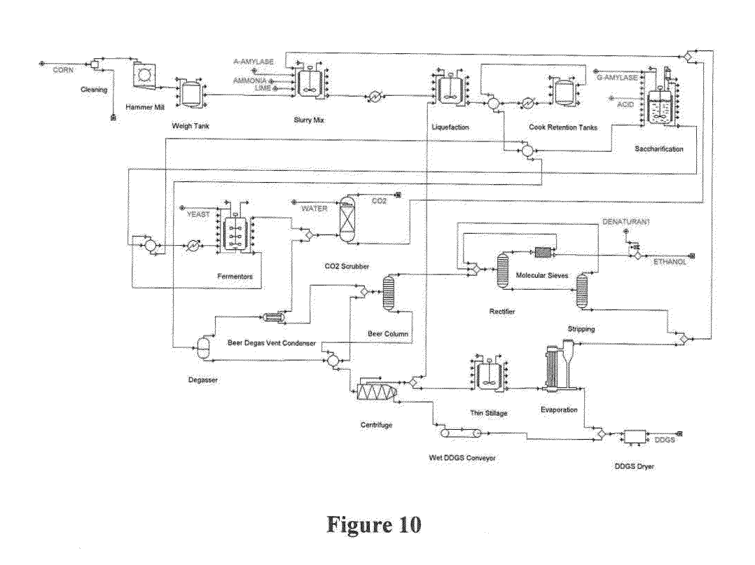

FIG. 11 shows a general flow chart for oil recovery after fermentation as described below. Dashed boxes represent two separate locations in the process that oil recovery could be accomplished.

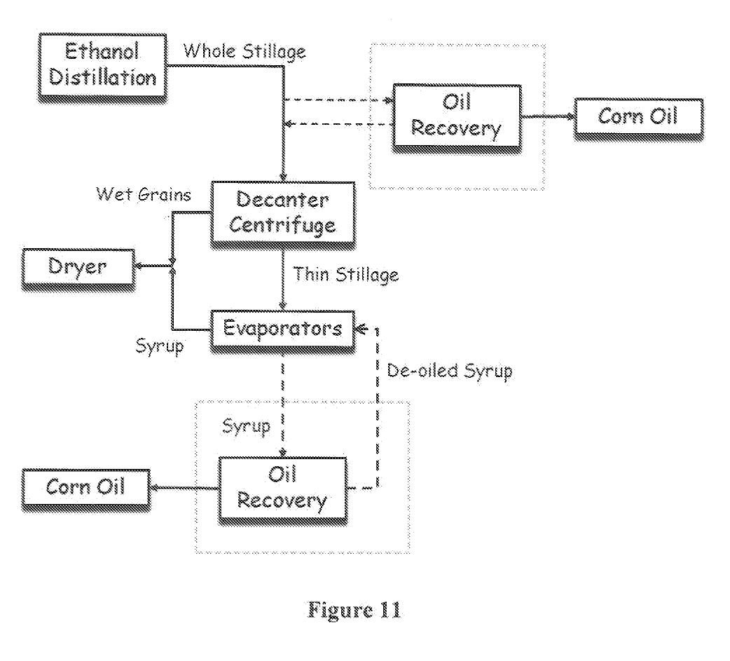

### DETAILED DESCRIPTION OF THE INVENTION

Disclosed are methods for obtaining oil from maize, involving grinding maize kernels to form flour, adding water to the flour to form a slurry, and incubating the slurry with α-amylase for about 10 minutes to about 180 minutes at a temperature of about 75° to about 120° C. and at a pH of about 3 to about 7 to form a mash, cooling the mash to about 15° C. to about 40° C. and adding a nitrogen source, glucoamylase, yeast, acid protease, and cell-wall polysaccharide-degrading enzymes to form a beer containing ethanol and oil, wherein the beer has a pH of about 3 to about 7, and recovering oil from the beer.

The corn may be, for example, whole kernel or flaked corn. Moisture content of feed material should be about 0 to about 14% by weight (e.g., 0 to 14% by weight). Although virtually any type and quality of grain can be used to produce ethanol, the feedstock for these processes is typically a corn known as “No. 2 Yellow Dent Corn.” The “No. 2” refers to a quality of corn having certain characteristics as defined by the National Grain Inspection Association and USDA Grain Inspection, Packers and Stockyards Administration, as is known in the art. “Yellow Dent” refers to a specific type of corn as is known in the art.

Dry grinding conditions would generally be the same as used by the corn dry grind ethanol industry. Dried whole corn kernels are inputted to a dry grind processing step in order to grind them into a flour (meal). Corn particle size data typically used in commercial corn to ethanol facilities is as given in Rausch, K. D., et al., Particle Size Distributions of Ground Corn and DDGS from Dry Grind Processing, Transactions of the ASAE, 48 (1): 273-277 (2005). Corn is ground so that greater than 85% passes through a 2.0 mm screen (Rausch et al.). However, we have found that the finer you grind the corn (particularly the germ) the more oil you can recover from the corn; preferably the flour contains particles of about 2 to about 0.25 mm (e.g., 2 to 0.25 mm), preferably about 1.5 to about 0.25 mm (e.g., 1.5 to 0.25 mm), more preferably about 0.6 to about 0.25 mm (e.g. 0.6 to 0.25 mm). The ground meal or flour is mixed with water to create a slurry, and a commercial enzyme called alpha-amylase is added. This slurry is then heated to about 75° to about 120° C. (e.g., 75° to about 120° C.), preferably about 85° to about 115° C. (e.g., 85° to about 115° C.), more preferably about 90° to about 110° C. (e.g., 90° to 110° C.), with or without jet cooking, at a pH of about 3 to about 7 (e.g., 3 to 7), preferably about 4 to about 7 (e.g., 4 to 7), more preferably about 5 to about 6.5 (e.g., 5 to 6.5) for about 10 to about 180 minutes (e.g., 10 to 180 minutes), preferably about 20 to about 100 minutes (e.g., 20 to 100 minutes), more preferably about 30 to about 90 minutes (e.g., 30 to 90 minutes) in order for alpha-amylase to hydrolyze the gelatinized starch into maltodextrins and oligosaccharides (chains of glucose sugar molecules) to produce a liquefied mash or slurry which has about 15 to about 50% by weight (e.g., 15 to 50% by weight) total solids content, preferably about 20 to about 40% by weight (e.g., 20 to 40% by weight), more preferably about 25 to about 35% by weight (e.g., 25 to 35% by weight), most preferably about 32 to about 33% by weight (e.g., 32 to 33% by weight). This is followed by separate saccharification and fermentation steps, although in most commercial dry grind ethanol processes saccharification and fermentation occur simultaneously (this step is referred to in the industry as “Simultaneous Saccharification and Fermentation” (SSF)). During saccharification the liquefied mash is cooled to about 15° to about 45° C. (e.g., 15° to 45° C.), preferably about 25° to about 40° C. (e.g., 25° to 40° C.), more preferably about 30° to about 35° C. (e.g., 30° to 45° C.), and after reducing the pH to about 3 to about 7 (e.g., 3 to 7), preferably about 3 to about 5 (e.g., 3 to 5), more preferably about 3.5 to about 4.5 (e.g., 3.5 to 4.5) a commercial enzyme known as gluco-amylase (e.g. DISTILLASE® SSF from DuPont Industrial Biosciences) is added. In our process, we also add at least one acid protease and cell-wall polysaccharide-degrading enzymes (e.g., cellulases and hemicellulases since cellulase enzymes are not really pure and do contain some hemicellulases) in order to improve oil recovery; ideally the enzymes would be added as the fermentor is being filled. A nitrogen source such as urea is also typically added to supply the yeast with a supplemental source during the fermentation process. The nitrogen source is typically added before liquefaction but could be added later in the process. The gluco-amylase hydrolyzes the maltodextrins and short-chained oligosaccharides into single glucose sugar molecules to produce a liquefied mash, which is also a “fermentation feed” when SSF is employed. During fermentation, a common strain of yeast (Saccharomyces cerevisiae) is added to metabolize the glucose sugars into ethanol and CO₂. Both saccharification and SSF can take as long as about 30 to about 90 hours (e.g., 30 to 90 hours), preferably about 40 to about 80 hours (e.g., 40 to 80 hours), more preferably about 50 to about 75 hours (e.g., 50 to 75 hours) but could be done for longer or shorter periods of time. Upon completion, the fermentation broth (“beer”) will contain about 17% to about 18% ethanol (volume/volume basis) (e.g., 17 to 18%), plus soluble and insoluble solids from all the remaining grain components. The final ethanol content is based on the starting concentration of starch and the conversion efficiency of the enzymes and the yeast, and may be higher or lower.

The beer is then processed to strip the ethanol from the beer and the ethanol is further purified in a series of distillation columns. The whole stillage is the stream produced after the removal of the ethanol from the beer. The whole stillage stream is separated in decanter centrifuges to separate the solids (wet grains) and the liquid (thin stillage) portions. The thin stillage stream is concentrated by evaporation to produce syrup (condensed distillers soluble (CDS)). The current process of recovery of oil typically begins after the thin stillage stream has been concentrated to produce the syrup or condensed distillers solubles. This syrup is then treated with thermal and/or chemical treatments to help release the emulsified oil within the stream. Following these additional treatments, the syrup is again centrifuged to recover the free oil. The chemical treatments are typically proprietary compounds that are designed to release the emulsified oil and are available from several different suppliers. After removal of the oil from the syrup by centrifugation, the syrup can be mixed with the wet grains for drying into a low-fat distiller's dried grains with solubles (DDGS).

An alternative process for oil recovery from the thin stillage stream uses additional centrifuges prior to the decanter to effectively wash the whole stillage stream to aid recovery of oil trapped within the solids portion of the whole stillage. The thin stillage is then evaporated to syrup and treated as above to remove the oil.

Recovery of oil is well known in the art; see, for example, Moreau, R. A., et al., Aqueous extraction of corn oil after fermentation in the dry grind ethanol process, In: Green Oil Processing, Farr, W., and A. Proctor, editors, AOCS Press, Urbana, Ill., pages 53-70 (2012).

The oil recovery yield from the current art process is typically only 25% of the oil content of the incoming corn. Without the implementation of the thermal and mechanical treatments of the thin stillage stream, little or no free oil would be recoverable.

The process we developed utilizes additional enzymes (e.g., acid protease and cell-wall polysaccharide-degrading enzymes such as cellulases and hemicellulases) which are added just before or during fermentation. Following fermentation, the ethanol is stripped from the beer to produce a modified whole stillage stream. The properties of this stream are altered because of the enzyme treatment during fermentation. Then the whole stillage stream would be processed just as described above: first by using the decanter to separate wet grains and thin stillage, next to concentrate the thin stillage into a syrup, and then treated with chemical and/or thermal treatments, followed by centrifugation to recover the corn oil. This new process allows for increased recovery of oil using centrifugation. Recoveries from the enzymatic treatments are significantly greater than the 25% reported in conventional processes and are about 40% or higher (e.g., 40% or higher), preferably about 40% to about 55% (e.g., 40% to 55%.

As noted above, in our process we also add acid protease and cell-wall polysaccharide-degrading enzymes (e.g., cellulases and hemicellulases) in order to improve oil recovery. The acid protease may be any acid protease known in the art; for example FERMGEN™ from DuPont Industrial Biosciences. The cellulase may be any cellulase known in the art; for example GC220 from DuPont Industrial Biosciences. At least one of the components of the blend of acid protease and cellulase (e.g., GC220 and FERMGEN™) should represent about 80% by weight (e.g., 80%) of the combination, preferably about 60% by weight, (e.g., 60%) and more preferably about equal parts by weight. Enzyme concentration (acid protease and/or cellulase) would be from as little as about 0.25 kg to about 15 kg per metric ton of corn on a dry weight basis (e.g., 0.25 to about 15 kg per metric ton), preferably about 0.5 to about 10 kg per metric ton (e.g., 0.5 to about 10 kg per metric ton), more preferably about 0.5 to about 5 kg per metric ton (e.g., 0.5 to 5 kg per metric ton).

The specific amount added would be based on the amount of increase in oil recovery wanted. Reaction time can potentially be reduced using increased levels of enzymes. Alpha- and gluco-amylase are currently used at levels of about 1 kg/MT in order to convert the starch in the corn kernels into glucose so that the yeast can then convert into ethanol.

It is within the skill of one skilled in the art to optimize the amount of enzymes. The incubation time can be increased so less enzyme can be used.

It is also within the skill of one skilled in the art to determine which enzymes can be successfully utilized. Selection of other enzymes that could be used in this process would need to consider activity and stability under the specific conditions used. Such enzymes would need to have the ability to disrupt the oil bodies so that the oil would be released from within the oil bodies and from possible association with the oil body membranes. Enzymes that would disrupt the oil body membrane could be other proteases that degrade the oleosins (structural proteins) that stabilize the oil body membranes or phospholipases that could disrupt the phospholipid monolayer in the membrane of the oil bodies that surround the oil. The enzymes would also need to have the ability to release the oil from the barriers within the cell wall matrix. This release may require various individual enzymes or combinations of enzymes depending on the specific structure of the cell wall, and likely a mixture of cell-wall polysaccharide-degrading enzymes (cell wall degrading enzymes) such as cellulases, hemicellulases, xylanases, pectinases, and beta-glucanases may be required. The enzymes could also prevent the stabilization of emulsions that could be formed once the oil is freed from the oil bodies. Components of the kernel such as corn fiber gum (an arabinoxylan) or zein (a hydrophobic protein) could interact with the freed oil and form stable emulsions. Enzymes that hydrolyze these emulsion-stabilizing components would release emulsified oil or prevent it from becoming emulsified.

Unless defined otherwise, all technical and scientific terms used herein have the same meaning as commonly understood by one of ordinary skill in the art to which the invention belongs. The term “about” is defined as plus or minus ten percent; for example, about 100° F. means 90° F. to 110° F. Although any methods and materials similar or equivalent to those described herein can be used in the practice or testing of the present invention, the preferred methods and materials are now described.

The following examples are intended only to further illustrate the invention and are not intended to limit the scope of the invention as defined by the claims.

### EXAMPLES

Materials and Methods. Enzymes: The enzymes used were gifts of DuPont Industrial Biosciences. SPEZYME® Fred (thermostable alpha-amylase) and OPTIDEX®L-400 (glucoamylase) were used to prepare the corn mash as described below. SPEZYME® CP (cellulase), GC 220 (cellulase), FERMGEN™ (acid protease), MULTIFECT® Xylanase (xylanase/cellulase). ACCELLERASE® 1500 (cellulase/hemicellulase) and ACCELLERASE® XY (xylanase) were used in oil recovery experiments as described below.

Oil Content: The oil content of the corn used for fermentations was determined using hexane extraction with a Dionex ASE system as previously described (Johnston et al., Journal of the American Oil Chemists Society 82:6030608 (2005); Moreau, R. A., et al., Journal of Agricultural and Food Chemistry, 44:2149-2154 (1996)).

Flour Grinding and Analysis: Yellow dent corn was ground in a plate mill (model G2, Bunn, Springfield, Ill.) so the flour produced would pass through a 2 mm screen. The plate gap was altered for experiments where grind size was evaluated. Ground corn particle sizing was evaluated using a sieve shaker (Great Western Manufacturing, Leavenworth, Kans.) with seven sieves (SSBC 14, 20, 24, 34, 44, 54, and 74) and a pan. The particle size range was determine by the screen size used, and was reported as percent of a 100 g sample retained on the screen: 1.6 mm and larger (1.6 mm), 1.0 to 1.6 mm (1.0 mm), 0.87 to 1.0 mm (0.87 mm), 0.58 to 0.87 mm (0.58 mm), 0.44 to 0.58 mm (0.44 mm), 0.37 to 0.44 mm (0.37 mm), 0.25 to 0.37 mm (0.25 mm), and less than 0.25 mm, respectively. The ground corn was dried overnight at 55° C. to reduce clumping prior to sieving as described by Rausch et al (Rausch, K. D., et al., Transactions of the ASAE, 48:273-277 (2005)). Moisture content of the flour was determined using AOAC Official Method 930.15 (AOAC Official Method 930.15, Official Methods of Analysis of AOAC International, 18th ed, AOAC International, Gaithersburg, Md. (2005)).

Mash Preparation: The appropriate amount of corn flour (adjusted for moisture content) and water were added to a beaker to make a 30% total solids solution and the total mass was measured to later compensate for water addition due to evaporation. Using a mechanical mixer, the pH was adjusted to 5.8 with 1 M HCl. Alpha-amylase (SPEZYME® FRED, DuPont Industrial Biosciences) was added at a dosage of 0.5 mL per kg of mash (2 kg/MT dry corn) and the slurry was heated to 95° C. and held for 60 min using a hot plate. The mash was then cooled to 30° C. and urea was added (400 ppm Nitrogen). The pH was then adjusted to 4.5 with 1 M HCl and glucoamylase (OPTIDED® L-400, DuPont Industrial Biosciences) added at a dosage of 0.4 mL per kg of mash (1.6 kg/MT dry corn). Water was added as necessary to compensate for evaporation losses and active yeast was added (1.1 gram per kg of mash) to start the fermentation (Red Star Ethanol Red, Fermentis).

Oil Recovery: A 30% solids corn mash was prepared as described. Four hundred grams of the mash, already containing yeast, was distributed into each pre-weighed 500 mL Erlenmeyer flasks equipped with rubber stoppers and 21 gauge needles to vent CO₂ produced during fermentation. The appropriate dose of enzyme was added to each flask and a final flask weight was measured. Flasks were incubated with shaking at 200 rpm for 72 hours at 30° C. and periodically weighed to determine loss due to CO₂ production.

After fermentation, the final flask mass was measured and a small (1 mL) sub-sample was removed for HPLC analysis. The entire contents were then transferred into a 600 mL beaker and heated to 90° C. with stirring. Samples were concentrated from the initial volume of about 350 mL to approximately 250 mL in order to approximate the stripping of ethanol from the beer after fermentation. The beaker contents were then transferred into a tared 250 mL centrifuge bottle and cooled to room temperature. Bottles were centrifuged for 10 min at 2000×g in a swinging bucket rotor. The oil and emulsion layer on the surface were transferred with a pipette to a 250 mL beaker in order to minimize losses that would occur by decanting. After the oil and emulsion layers were transferred, the liquid (approximately equivalent to the fraction called thin stillage) was decanted into the same beaker. The weight of the bottle with the pellet remaining was measured. The thin stillage (approximately 150 ml) was then heated to 90° C. and concentrated to about 45 mL in order to produce the equivalent of syrup (also know in the industry as Condensed Distillers Solubles (CDS)). The syrup was transferred into 50 mL centrifuge tubes. The centrifuge tubes were cooled to room temperature and centrifuged for 20 min at 2400×g.

The oil and emulsion layers were again removed with a pipette and transferred into 15 mL centrifuge tubes. Adding this final centrifugation step further concentrated the free oil so that it could be quantitatively measured. Additional liquid was transferred from the 50 mL tubes to make the final volumes about 12 mL in each 15 mL tube. These tubes were then centrifuged for 20 min at 4000×g. Tubes were visually compared to determine the volume of the free oil and of the emulsion layer directly below. The free oil was then carefully removed using a pipet into small tared glass tubes and the weight of the free oil recovered was measured.

HPLC Analysis: The small sub-sample from the shake flask was centrifuged (Eppendorf 5415D, at 16,000×g) and the supernatant filtered through a 0.2 um filter. The sample was then analyzed using an Agilent 1200 HPLC (Santa Clara, Calif.) equipped with a refractive index detector and an ion exclusion column (Aminex HPX-87H, Bio-Rad, Hercules, Calif.). The column was maintained at 65° C. and 5 mM sulfuric acid at 0.6 mL/min used for elution. The column was calibrated using analytical standards of maltodextrins (DP4+), maltotriose (DP3), maltose, glucose, fructose, succinic acid, lactic acid, acetic acid, glycerol, methanol and ethanol. Samples were filtered through 0.22 um syringe filters (Acrodise, PALL Life Sciences, MI) and injected (5 uL). The results were analyzed using the Agilent ChemStation software. Results reported are the average of duplicate injections.

Results and Discussion. Oil Recovery Method Development: The method developed and described above was intended to simulate, at the bench scale, the general process used in ethanol facilities that recover post fermentation oil. No organic solvents (such as hexane or methylene chloride) were used in the method as these could artificially increase oil recovery. The small initial oil content of the corn kernel, the small amount released during normal processing, and the unavoidable losses on glassware transfers and manipulations made method development difficult. Our preliminary experiments using a 100 g mash scale protocol were not successful at achieving reproducible increases in oil yields. We determined that a larger (400 g mash) fermentation would be a sufficient amount to accurately measure oil recovery and to also allow ethanol removal, solids separation and liquid concentration steps to be done. This scale was tested to determine reproducibility and was surprisingly found to give acceptable performance with reproducible results. Improved reproducibility was ultimately achieved through weighing the free oil rather than estimating oil volume.

It should be noted that the free oil recoveries obtained with the lab scale process were relatively lower than the oil yields reported in commercial processing facilities where about 25% of the total oil is recovered. Without being bound by theory, it is believed that the differences were due partially to the milder processing conditions (e.g., lower temperature centrifugation and low shear transfers) used with the lab scale recovery process; oil yield reductions may have also been due to losses during transfers or because the lab process only recovered clear free oil.

Enzyme Screening: Several commercial enzyme preparations were screened for their ability to increase oil recovery. Enzymes were selected based on pH compatibility with fermentation conditions. Cell wall degrading preparations (cellulases, hemicellulases and xylanases) were selected along with proteases. Control experiments in the absence of enzyme were also done with each batch of enzymes tested. The masses of the free oil recoveries were evaluated relative to the total oil content of the corn used in the fermentation as determined by hexane extraction. The results were reported as a percentage of the total oil in the mash as determined by hexane extraction of the corn flour.

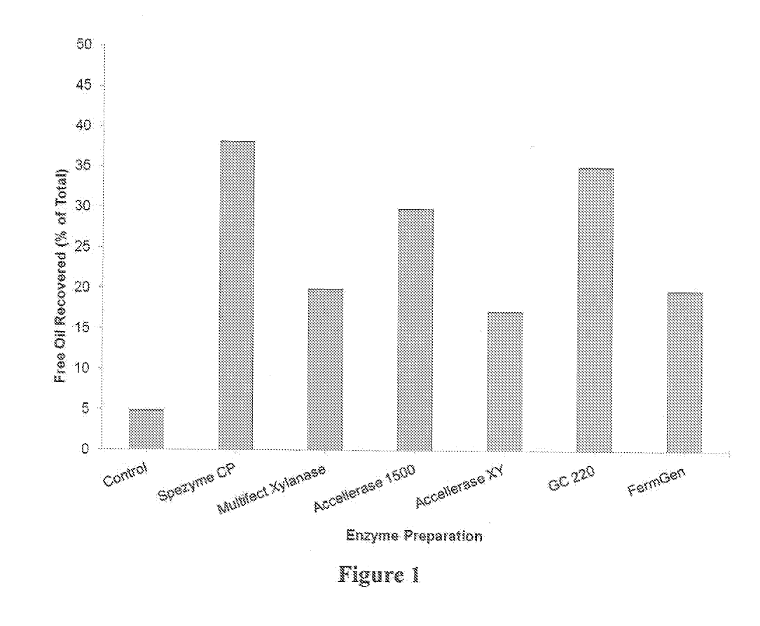

Screening results using an enzyme dose equivalent to 10 kg per MT of dry corn are shown in FIG. 1. In our preliminary screening experiment, the enzymes were screened at a much lower level (equivalent to about 1.0 kg per MT) but did not show a clear increase relative to the control (results not shown). It was only when the higher enzyme dose was used that all of the preparations tested exhibited a measurable improvement in the amount of free oil recovered relative to the control as shown in FIG. 1. SPEZYME CP and GC220 surprisingly gave the most significant increases relative to the control and were chosen for further study; unfortunately we did not have time to conduct additional experiments with Accellerase 1500 which can also be utilized in our process.

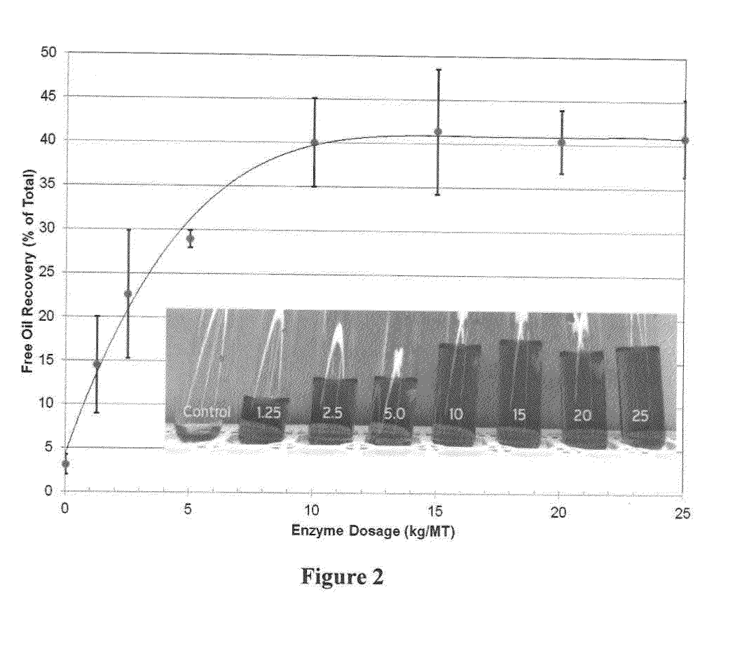

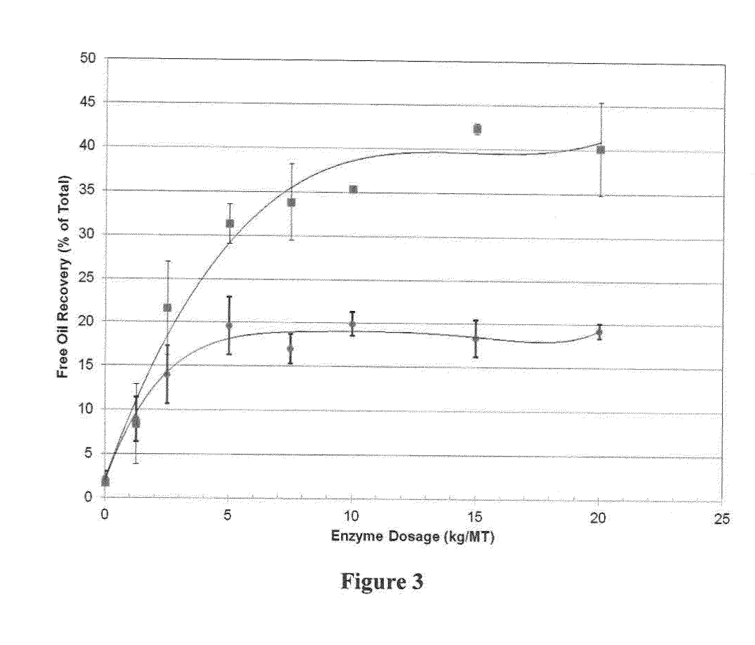

Enzyme Concentration Effects: Enzyme dosing experiments were conducted using the top two enzyme preparations from the screening studies, GC 220 and SPEZYME® CP. Both preparations have significant cellulase activity and were surprisingly found to produce significant increases in free oil relative to the control. The oil recoveries from experiments using increasing doses of SPEZYME® CP and GC 220 are shown in FIGS. 2 and 3 respectively (the FERMGEN™ dose response also shown in FIG. 3 will be discussed below). The results showed that increasing amounts led to increased oil recovery up to a point where additional enzyme resulted in little or no further increase. With each enzyme, the final free oil recoveries were above 40%. However, more than 50% of the oil was still trapped in the solids removed during centrifugation or in the particle/emulsion layer just below the free oil. The higher enzyme concentrations did surprisingly yield increased free oil recoveries relative to typical ethanol plant recoveries. However, the lower enzyme levels resulted in free oil yields less than what is typically reported in commercial facilities. As previously stated, it is unclear if the lower oil yields were due to the milder processing conditions or transfer losses that inevitably occur during recovery of the (sticky) clear free oil in a small lab scale model system. What was clear was that a significant amount of oil was still trapped in the solids fraction separated during the first centrifugation.

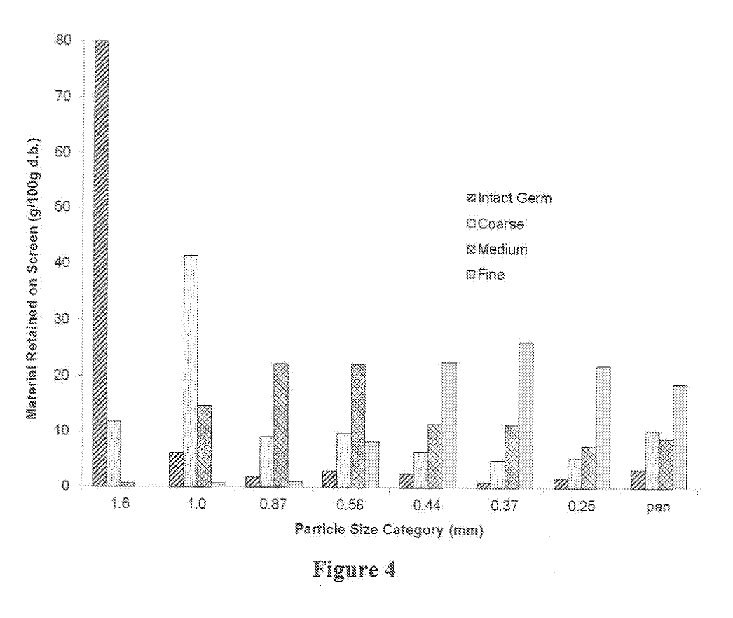

Effects of Initial Particle Size: We tested whether particle size reduction may be a possible method to improve the oil release during ethanol production. Reduction of the initial corn particle size was tested to determine if oil recovery could be improved further. Corn was ground to three different particle sizes distributions (coarse, medium and fine (FIG. 4); the coarse particles were generally about 1.5 mm or smaller, the medium particles were generally 1.0 mm or smaller, the fine particles were generally about 0.5 mm or smaller, and the intact germ particles were generally greater than 1.6 mm) using a disk mill and a fourth sample was ground using a de-germination mill (particle size distribution shown in FIG. 4). The de-germination mill left the germ (the location of more than 85% of the corn oil) intact, therefore significantly limiting accessibility of the oil vesicles; the endosperm was also relatively coarse when ground by this method, and resulted in decreased final ethanol yields in these samples (data not shown). The particle size distribution of the four different corn flours is shown in FIG. 4. Additionally, one sample was further reduced in particle size using a Polytron homogenizer after the corn had been processed through the liquefaction procedure and cooled. Homogenization was performed using a 20 mm standard homogenization generator at high speed for 10 min with a 1500 mL mash preparation made using the finely ground corn flour. The resulting mash had a much smoother consistency relative to any of the other mash preparations. Particle size analysis was not done on this preparation.

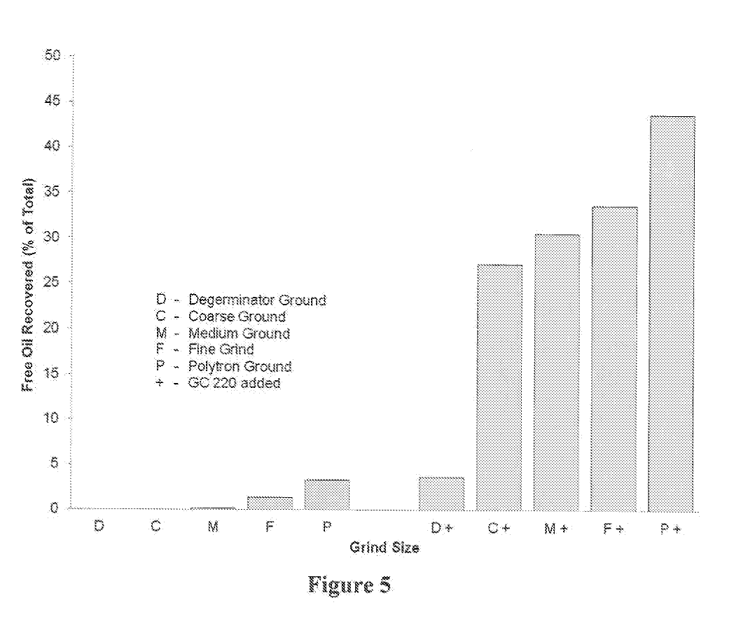

The post fermentation oil recoveries for the differently ground flours, with and without the addition of GC 220, are shown in FIG. 5. The free oil recoveries without enzyme addition did showed a correlation between increasing oil with decreasing particle size; however, the overall free oil yields were relatively low, with no free oil recoverable from the coarse or the intact germ flours. The same surprising trend of increasing oil recovery with decreasing particle size was observed with the enzyme treated samples. Surprisingly the overall recoveries were significantly higher with the enzyme treatments relative to the untreated flours with the exception of the intact germ flour. Using intact or coarse germ rather than finely ground germ clearly resulted in a reduction in the free oil recovery; this reduction strongly suggested the surprising importance that particle size reduction (specifically of the germ) had on the release of oil from the corn solids beneficially aiding in the recovery of free oil after fermentation.

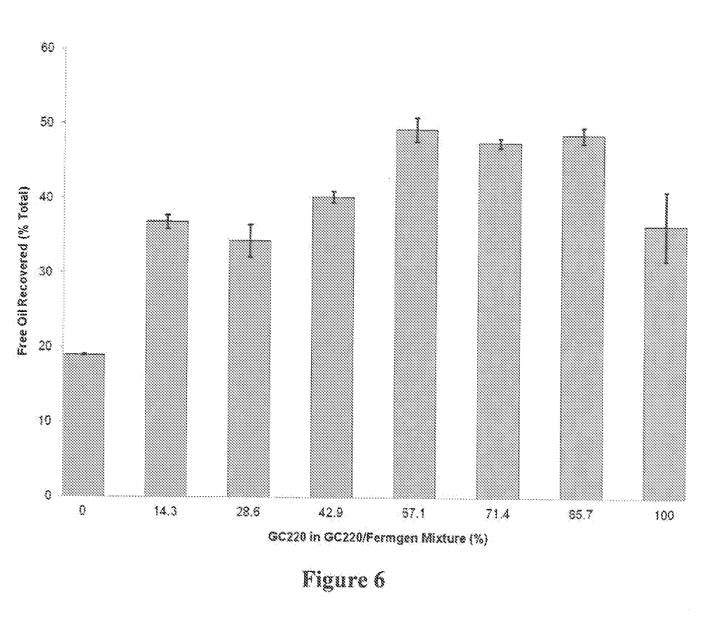

Effects of Protease Addition: Initial studies utilizing mixtures of enzymes to try to improve the overall oil recovery surprisingly met with little success. In most cases the results did not yield more oil relative to the individual preparations (results not shown). We did, however, discover that mixing an acid protease (e.g., FERMGEN™) with a cell wall degrading preparation (e.g., cellulase like GC 220) surprisingly resulted in what appeared to be a synergistic effect. These results are shown in FIG. 6. In these experiments, a fixed amount (7 kg/MT dry corn) of enzyme was added to each flask but the ratio of FERMGEN™ to GC 220 was varied. The results clearly show that when the ratio was predominately GC 220 when FERMGEN™ was added the oil yield surprisingly increased above the level of GC 220 alone. Surprisingly this was also the case when the mixture was predominately FERMGEN™ and GC 220 was added; however, recoveries were somewhat reduced. As the ratio increased in FERMGEN™ (GC 220 decreasing), the yields decreased but were still greater than the yields of FERMGEN™ alone.

To confirm that the FERMGEN™ levels being used were not at a saturation point (where a reduction in enzyme would not produce a corresponding reduction in oil recovery), a dosing response curve for FERMGEN™ was also constructed (FIG. 3) and it surprisingly demonstrated a significant difference in response relative to the other enzymes tested. Using GC 220 alone (as well as Spezyme CP), surprisingly the maximum free oil obtained was slightly above 40% of the total oil in the corn; however, with FERMGEN™ alone the maximum was surprisingly only about 20%. This was unexpected and showed that the levels used in the mixture experiment were too high to confirm that we were observing a synergistic effect with the two enzymes. The results did however suggest the possibility that the enzymes were releasing oil using different mechanisms. Without being bound by theory, if they were using the same mechanism, the protease would have most likely continued to increase yield with the increasing dose until it reached the same yield as GC 220 and Spezyme CP.

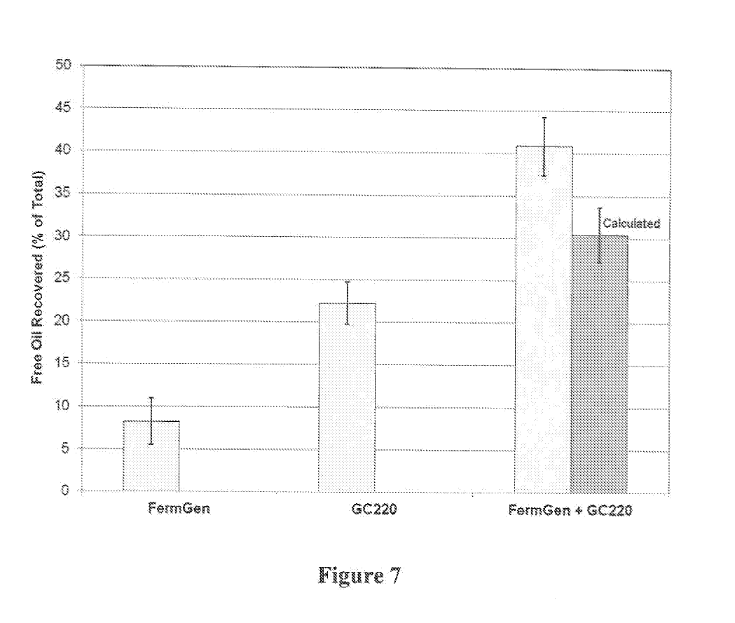

Using levels well below the saturation point for each enzyme, a second experiment was done to confirm the synergistic observation. Triplicate flasks were prepared from the same mash using 1 kg/MT FERMGEN™, 2.5 kg/MT GC 220, and the mixture of the two at these same levels. The free oil results are shown in FIG. 7 and surprisingly and clearly show, with statistical significance, a synergistic response with the enzyme mixture. A slightly greater than 10% increase in free oil was surprisingly observed for the enzyme mixture when compared to the addition of the two used independently (labeled “calculated” on FIG. 7).

Ethanol production: Fermentation rates determined by weight loss and final ethanol values measured by HPLC were measured for all control and enzyme addition experiments. Ethanol yield values were surprisingly found to increase on average with increasing levels of GC220 and Spezyme CP, and the increase was statistically significant at the highest levels of enzyme addition relative to the control (results not shown). FERMGEN™ addition surprisingly did not show significant increases in final ethanol yield but did show significant increases in fermentation rates relative to the controls. This indicated that increased conversion of glucose to ethanol and carbon dioxide was not the result of more glucose being made available but rather the improved utilization by the yeast of the available nutrients. Ethanol levels were measure at the end of the 72 hour fermentations to confirm that there was no inhibition created by the enzyme addition. If ethanol levels had been measured at an earlier time point, the results would have been significantly different due to the increased fermentation rate. At earlier time points, analysis of faster fermentations of the FERMGEN™ treatments would have produced increased concentrations of ethanol relative to the untreated samples. As the glucose concentrations were exhausted, conversion slowed and gave time for the slower, non-FERMGEN™ treated samples to catch up and reach equivalent ethanol concentrations.

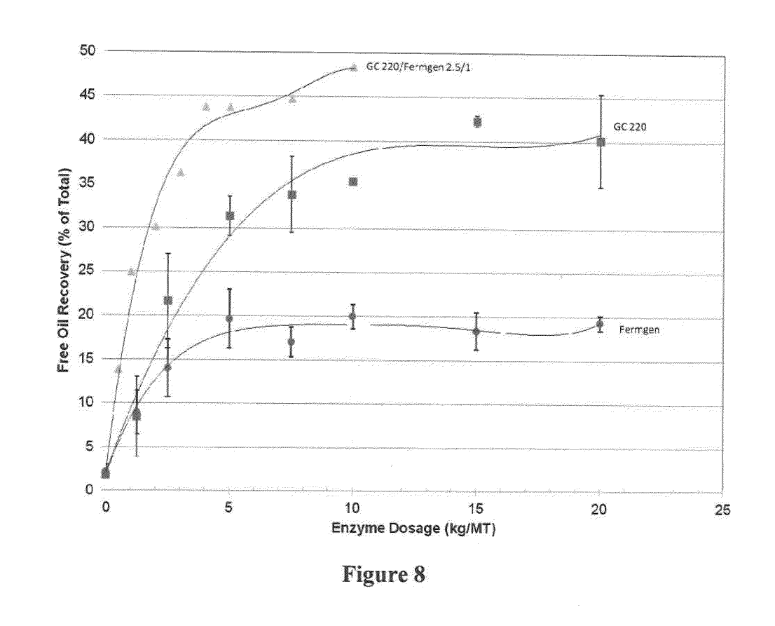

The synergistic effects demonstrated in FIG. 7 showed results for a single dosage of enzyme at a ratio of 2.5:1 (GC220: FERMGEN™). To see how this mixture of enzyme would compare to the individual preparation, the same mixture was tested over a range of enzyme loadings. FIG. 8 shows the results and includes the data from the individual preparation as previously shown. It can easily be seen that the mixture of GC220 and FERMGEN™ together surprisingly resulted in significantly more free oil recovered relative to the individual preparations when used at equivalent loadings.

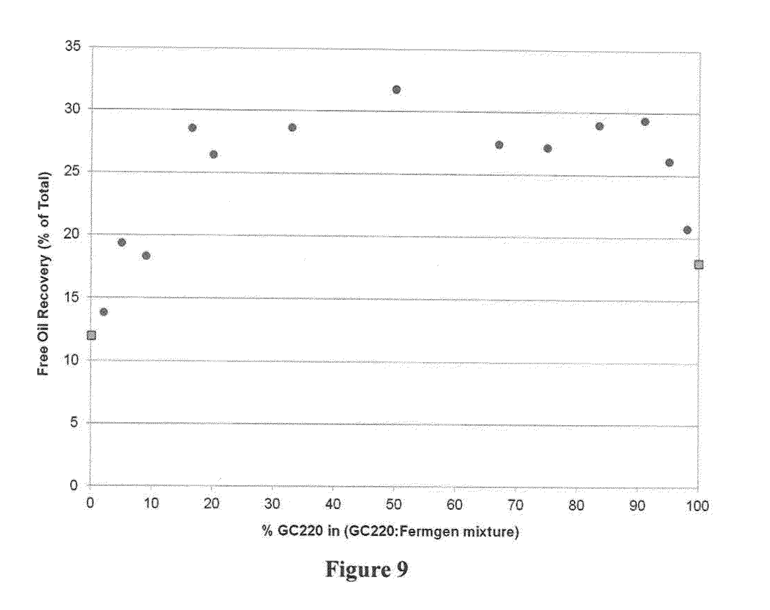

To further examine the effects of oil recovery with mixtures of FERMGEN™ and GC220, we prepared enzyme mixtures using different ratios of each enzyme. These mixtures were then added at equal quantities (2 kg/MT) during fermentation. The 2 kg/MT level was chosen so that increases or decreases in oil recovery could easily be measured. The goal of these experiments was to determine if a particular enzyme mixture improved or decreased free oil recovery. FIG. 9 shows the results for these experiments. The square data points represent expected yields with GC220 or FERMGEN™ alone at the 2 kg/MT level. The data clearly showed that with mixtures of just a few percent of the other enzyme that the free oil recovery yields were surprisingly increased. The highest yield increase surprisingly appeared to be at or near a mixture of equal parts for each enzyme; however, incorporation of as little as 2% by volume of the other enzyme surprisingly showed an increase relative to the pure enzyme preparation. At incorporation of 5-10% by volume, free oil recoveries were surprisingly and markedly improved.

### CONCLUSION

The above demonstrated that only certain enzymes, when added at a sufficient level during laboratory scale for corn to ethanol fermentations, surprisingly improved the amount of free oil that can be recovered (e.g., by centrifugation or flotation) after fermentation. Particle size reduction of the corn before fermentation was also found to surprisingly improve free oil recovery with added enzymes and the reduction of the germ particles was surprisingly found to be particularly important. The free oil recoveries obtained using acid protease/cellulase enzyme mixtures, resulted in oil recoveries that were surprisingly higher than the values typically reported by ethanol production facilities recovering oil without adding these enzymes during fermentation. The use of a specific mixture of an acid protease (e.g., FERMGEN™) with a cellulase (e.g., GC 220) was surprisingly found to exhibit a synergistic behavior relative to free oil recovery. This surprising behavior resulted in a reduction of the total amount of enzyme needed and could help to improve the economics of the enzyme assisted oil recovery process. To our knowledge this is the first report showing that acid protease and cellulase enzymes added during fermentation can be used to improve the downstream oil recovery process.

All of the references cited herein, including U.S. patents, are incorporated by reference in their entirety. Also incorporated by reference in their entirety are the following references: Moreau, R. A., et al., Aqueous enzymatic oil extraction: A “Green” bioprocess to obtain oil from corn germ and other oil-rich plant materials, pages 101-120, in: ACS Symposium Series, G. Eggleston and J. R. Vercellotti, eds (2007); Moreau, R. A., et al., Journal of the American Oil Chemists' Society, 81:1071-1075 (2004); Yadav M. P., et al., Journal of Agricultural and Food Chemistry, 55 (15):6366-6371 (2007). Also incorporated by reference in their entirety are the following U.S. Pat. Nos. 8,168,037; 8,008,517; 8,008,516; 7,601,858; 7,608,729; 7,148,366; and 7,101,691.

Thus, in view of the above, there is described (in part) the following:

A method for obtaining oil from maize, comprising (or consisting essentially of or consisting of) grinding maize kernels to form flour, adding water to said flour to form a slurry, and incubating said slurry with α-amylase for about 10 minutes to about 180 minutes at a temperature of about 75° to about 120° C. and at a pH of about 3 to about 7 to form a mash, cooling said mash to about 15° C. to about 40° C. and adding a nitrogen source, glucoamylase, yeast, acid protease, and cell-wall polysaccharide-degrading enzymes to form a beer containing ethanol and oil, wherein said beer has a pH of about 3 to about 7, and recovering oil from said beer.

The above method, wherein said cell-wall polysaccharide-degrading enzymes are cellulases and hemicellulases.

The above method, wherein the concentration of said acid protease and cell-wall polysaccharide-degrading enzymes is about 0.25 kg to about 15 kg per metric ton of corn on a dry weight basis. The above method, wherein the concentration of said acid protease and cell-wall polysaccharide-degrading enzymes is about 0.5 to about 10 kg per metric ton of corn. The above method, wherein the concentration of said acid protease and cell-wall polysaccharide-degrading enzymes is about 0.5 to about 5 kg per metric ton of corn.

The above method, wherein said flour has particle size of about 2 to about 0.25 mm. The above method, wherein said flour has particle size of about 1.5 to about 0.25 mm. The above method, wherein said flour has particle size of about 0.6 to about 0.25 mm.

A method for obtaining oil from maize, comprising (or consisting essentially of or consisting of) grinding maize kernels to form flour, adding water to said flour to form a slurry, and incubating said slurry with α-amylase for about 10 minutes to about 180 minutes at a temperature of about 75° to about 120° C. and at a pH of about 3 to about 7 to form a mash, cooling said mash to about 15° C. to about 40° C. and adding a nitrogen source, glucoamylase, yeast, acid protease, and cell-wall polysaccharide-degrading enzymes to form a beer containing ethanol and oil, wherein said beer has a pH of about 3 to about 7, and removing said ethanol from said beer to form whole stillage, separating said whole stillage into wet grains and thin stillage, evaporating said thin stillage to form a syrup, and recovering oil from said syrup.

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

We claim:

1. A method for obtaining oil from maize, comprising grinding maize kernels to form flour, adding water to said flour to form a slurry, and incubating said slurry with α-amylase for about 10 minutes to about 180 minutes at a temperature of about 75° to about 120° C. and at a pH of about 3 to about 7 to form a mash, cooling said mash to about 15° C. to about 40° C. and adding a nitrogen source, glucoamylase, yeast, acid protease, and cell-wall polysaccharide-degrading enzymes to form a beer containing ethanol and oil, wherein said beer has a pH of about 3 to about 7, and recovering oil from said beer.

2. The method according to claim 1, wherein said cell-wall polysaccharide-degrading enzymes are cellulases and hemicellulases.

3. The method according to claim 1, wherein the concentration of said acid protease and cell-wall polysaccharide-degrading enzymes is about 0.25 kg to about 15 kg per metric ton of corn on a dry weight basis.

4. The method according to claim 1, wherein the concentration of said acid protease and cell-wall polysaccharide-degrading enzymes is about 0.5 to about 10 kg per metric ton of corn.

5. The method according to claim 1, wherein the concentration of said acid protease and cell-wall polysaccharide-degrading enzymes is about 0.5 to about 5 kg per metric ton of corn.

6. The method according to claim 1, wherein said flour has particle size of about 2 to about 0.25 mm.

7. The method according to claim 1, wherein said flour has particle size of about 1.5 to about 0.25 mm.

8. The method according to claim 1, wherein said flour has particle size of about 0.6 to about 0.25 mm.

9. A method for obtaining oil from maize, comprising grinding maize kernels to form flour, adding water to said flour to form a slurry, and incubating said slurry with α-amylase for about 10 minutes to about 180 minutes at a temperature of about 75° to about 120° C. and at a pH of about 3 to about 7 to form a mash, cooling said mash to about 15° C. to about 40° C. and adding a nitrogen source, glucoamylase, yeast, acid protease, and cell-wall polysaccharide-degrading enzymes to form a beer containing ethanol and oil, wherein said beer has a pH of about 3 to about 7, and removing said ethanol from said beer to form whole stillage, separating said whole stillage into wet grains and thin stillage, evaporating said thin stillage to form a syrup, and recovering oil from said syrup.
